(19) United States
(12) Patent Application Publication
(10) Pub. No.: US 2010/0029685 A1
(43) Pub. Date: Feb. 4, 2010

(54) OXADIAZOLE AND THIADIAZOLE COMPOUNDS AND THEIR USE AS NICOTINIC ACETYLCHOLINE RECEPTOR MODULATORS

(75) Inventors: Bjarne H. Dahl, Lynge (DK); Dan Peters, Malmo (SE); Gunnar M. Olsen, Smorum (DK); Daniel B. Timmermann, Herlev (DK); Susanne Jorgensen, Frederiksberg (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/447,205

(22) PCT Filed: Oct. 24, 2007
(86) PCT No.: PCT/EP2007/061433

§ 371 Date: Jun. 23, 2009

Related U.S. Application Data
(60) Provisional application No. 60/854,078, filed on Oct. 25, 2006.

(30) Foreign Application Priority Data
Oct. 25, 2006 (DK) PA 2006 01380

(51) Int. Cl. A61K 31/4965; C07D 271/07; C07D 401/14; C07D 285/08; C07D 413/04; C07D 417/04; A61K 31/4439; A61K 31/4155; A61P 25/28; A61P 25/02

(52) U.S. Cl. 514/255.05; 548/132; 544/405; 548/130; 546/269.4; 546/268.7; 514/340; 514/342; 514/364; 514/361

(57) ABSTRACT

This invention relates to oxadiazolyl and thiadiazolyl derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### TECHNICAL FIELD

This invention relates to oxadiazolyl and thiadiazolyl derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exerts its biological effect via two types of cholinergic receptors, the muscarinic Acetyl Choline Receptors (mAChR) and the nicotinic Acetyl Choline Receptors (nAChR).

Nicotinic acetylcholine receptors (nAChRs) are pentameric ligand gated ion channels and widely distributed throughout the central (CNS) and peripheral (PNS) nervous systems. At least 12 subunit proteins, i.e. α2-α10 and β2-β4, have been identified in neuronal tissue. These subunits provide for a great variety of homomeric and heteromeric combinations that account for the diverse receptor subtypes. For example, the predominant receptor that is responsible for high affinity binding of nicotine in brain tissue has composition α4β2, while another major population of receptors is comprised of the homomeric α7.

Discovery of the important role played by nAChRs in several CNS disorders has called attention to these membrane proteins and to ligands able to modulate their functions. The existence of different subtypes at multiple levels has complicated the understanding of this receptor's physiological role, but at the same time has increased the efforts to discover selective compounds in order to improve the pharmacological characterization of this kind of receptor and to make safer the possible therapeutic use of its modulators.

Oxadiazolyl derivatives have been described for use as e.g. plant growth regulators, see e.g. U.S. Pat. No. 3,947,263, for use as herbicides, see e.g. U.S. Pat. No. 3,964,896, and for use as pesticides, see e.g. WO 98/57969. However, the oxadiazolyl and thiadiazolyl derivatives of the present invention have never been described, or their activity as modulators of the nicotinic receptors certainly never suggested.

### SUMMARY OF THE INVENTION

The present invention is devoted to the provision modulators of the nicotinic receptors, which modulators are useful for the treatment of diseases or disorders related to the nicotinic acetylcholine receptor (nAChR).

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and abuse liability and withdrawal symptoms caused by the termination of abuse of chemical substances, in particular nicotine.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention provides an oxadiazole or a thiadiazole derivative of Formula I

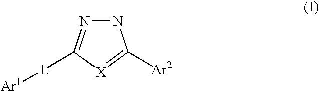

any of its isomers or any mixture of its isomers, an N-oxide, a prodrug, or a pharmaceutically-acceptable addition salt thereof, wherein

Ar¹ represents a phenyl, pyridinyl, pyridazinyl, pyrimidinyl or pyrazinyl group, which phenyl, pyridinyl, pyridazinyl, pyrimidinyl and pyrazinyl groups may optionally be substituted one or more times with substituents selected from the group consisting of alkyl, hydroxy, alkoxy, halo, haloalkyl, haloalkoxy, nitro, cyano, acetonitrile, amino-carbonyl (carbamoyl) and methylenedioxy;

Ar² represents alkyl-carbonyl-amino (acetamido), or a phenyl, furanyl, thienyl, isoxazolyl, thiazolyl or pyridinyl group, which phenyl, furanyl, thienyl, isoxazolyl, thiazolyl and pyridinyl groups may optionally be substituted one or more times with substituents selected from the group consisting of alkyl, halo, haloalkyl, haloalkoxy, nitro and cyano;

L may be absent (i.e. represents a single covalent bond) or present, and if present represents a linking group selected from CH₂, CH₂CH₂, S, S—CH₂, O, O—CH₂, SO₂ and SO₂CH₂; and

X represents O or S.

In a second aspect the invention provides pharmaceutical compositions comprising a therapeutically effective amount of the oxadiazole derivative of the invention, or a pharmaceutically-acceptable addition salt thereof, together with at least one pharmaceutically-acceptable carrier or diluent.

Viewed from another aspect the invention relates to the use of the oxadiazole derivative of the invention, or a pharmaceutically-acceptable addition salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the oxadiazole derivative of the invention.

Other objects of the invention will be apparent to the person skilled in the art from the following detailed description and examples.

### DETAILED DISCLOSURE OF THE INVENTION

### Oxadiazole Derivatives

In its first aspect the invention provides an oxadiazole or a thiadiazole derivative of Formula I

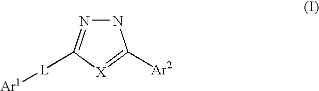

any of its isomers or any mixture of its isomers, an N-oxide, a prodrug, or a pharmaceutically-acceptable addition salt thereof, wherein

Ar¹ represents a phenyl, pyridinyl, pyridazinyl, pyrimidinyl or pyrazinyl group, which phenyl, pyridinyl, pyridazinyl, pyrimidinyl and pyrazinyl groups may optionally be substituted one or more times with substituents selected from the group consisting of alkyl, hydroxy, alkoxy, halo, haloalkyl, haloalkoxy, nitro, cyano, acetonitrile, amino-carbonyl (carbamoyl) and methylenedioxy;

Ar² represents alkyl-carbonyl-amino (acetamido), or a phenyl, furanyl, thienyl, isoxazolyl, thiazolyl or pyridinyl group, which phenyl, furanyl, thienyl, isoxazolyl, thiazolyl and pyridinyl groups may optionally be substituted one or more times with substituents selected from the group consisting of alkyl, halo, haloalkyl, haloalkoxy, nitro and cyano;

L may be absent (i.e. represents a single covalent bond) or present, and if present represents a linking group selected from CH₂, CH₂CH₂, S, S—CH₂, O, O—CH₂, SO₂ and SO₂CH₂; and

X represents O or S;

provided, however, that the compound is not
N-(5-Benzylsulfanyl-[1,3,4]thiadiazol-2-yl)-acetamide;
3-(5-(5-Nitro-furan-2-yl)-[1,2,4]oxadiazol-3-yl)-pyridine;
3-(5-(3-Nitro-phenyl)-[1,2,4]oxadiazol-3-yl)-pyridine; or
2-Acetamido-5-benzylthio-[1,3,4]thiadiazole.

In a preferred embodiment the compound of the invention is represented by Formula I, wherein Ar¹ represents a phenyl, pyridinyl, pyridazinyl, pyrimidinyl or pyrazinyl group, which phenyl, pyridinyl, pyridazinyl, pyrimidinyl and pyrazinyl groups may optionally be substituted one or more times with substituents selected from the group consisting of alkyl, hydroxy, alkoxy, halo, haloalkyl, haloalkoxy, nitro, cyano, acetonitrile, amino-carbonyl (carbamoyl) and methylenedioxy.

In a more preferred embodiment Ar¹ represents a phenyl, pyridinyl, pyridazinyl, pyrimidinyl or pyrazinyl group, which phenyl, pyridinyl, pyridazinyl, pyrimidinyl and pyrazinyl groups may optionally be substituted one or more times with substituents selected from the group consisting of alkyl, hydroxy, alkoxy, halo, nitro, cyano, acetonitrile, amino-carbonyl (carbamoyl) and methylenedioxy.

In an even more preferred embodiment Ar¹ represents a pyridinyl, pyridazinyl, pyrimidinyl or pyrazinyl group, which pyridinyl, pyridazinyl, pyrimidinyl and pyrazinyl groups may optionally be substituted one or more times with substituents selected from the group consisting of alkyl, hydroxy, alkoxy, halo, nitro, cyano, acetonitrile, amino-carbonyl (carbamoyl) and methylenedioxy.

In a still more preferred embodiment Ar¹ represents a pyridinyl, pyridazinyl, pyrimidinyl or pyrazinyl group, which phenyl, pyridinyl, pyridazinyl, pyrimidinyl and pyrazinyl groups may optionally be substituted one or more times with substituents selected from the group consisting of alkyl, in particular methyl, and halo, in particular fluoro or chloro.

In another more preferred embodiment Ar¹ represents a phenyl, pyridinyl, pyridazinyl or pyrazinyl group, which phenyl, pyridinyl, pyridazinyl and pyrazinyl groups may optionally be substituted one or more times with substituents selected from the group consisting of alkyl, hydroxy, alkoxy, halo, haloalkyl, haloalkoxy, nitro, cyano, acetonitrile and amino-carbonyl (carbamoyl).

In an even more preferred embodiment Ar¹ represents a phenyl, pyridinyl, pyridazinyl or pyrazinyl group, which phenyl, pyridinyl, pyridazinyl and pyrazinyl groups may optionally be substituted one or more times with substituents selected from the group consisting of alkyl, hydroxy, alkoxy, halo, nitro, cyano, acetonitrile and amino-carbonyl (carbamoyl).

In a still more preferred embodiment Ar¹ represents phenyl, optionally substituted one or more times with substituents selected from the group consisting of alkyl, hydroxy, alkoxy, halo, nitro, cyano, acetonitrile, amino-carbonyl (carbamoyl) and methylenedioxy.

In a yet more preferred embodiment Ar¹ represents phenyl, optionally substituted one or more times with substituents selected from the group consisting of alkyl, hydroxy, alkoxy, halo, nitro, cyano, acetonitrile and amino-carbonyl (carbamoyl).

In a further more preferred embodiment Ar¹ represents phenyl, optionally substituted with nitro, cyano, acetonitrile or amino-carbonyl (carbamoyl).

In a still further more preferred embodiment Ar¹ represents phenyl, optionally substituted two times with substituents selected from the group consisting of alkyl, hydroxy, halo, and cyano.

In a still further more preferred embodiment Ar¹ represents phenyl, optionally substituted three times with substituents selected from the group consisting of alkoxy, and halo.

In a still further more preferred embodiment Ar¹ represents phenyl, 3-fluoro-phenyl, 3-nitro-phenyl, 3-cyano-phenyl, 4-chloro-phenyl, 4-cyano-phenyl, 4-cyano-3-alkyl-phenyl, 3-cyano-4-alkyl-phenyl, 3-acetonitrile-phenyl, 5-chloro-2-hydroxy-phenyl, 5-trifluoromethyl-2-chloro-phenyl, 3-carbamoyl-phenyl, 4,6-dimethoxy-3-chloro-phenyl or 3,4-ethylenedioxy-phenyl.

In a still further more preferred embodiment Ar¹ represents phenyl, 3-nitro-phenyl, 3-cyano-phenyl, 4-cyano-3-alkyl-phenyl, 3-cyano-4-alkyl-phenyl, 3-acetonitrile-phenyl, 5-chloro-2-hydroxy-phenyl, 5-trifluoromethyl-2-chloro-phenyl, 3-carbamoyl-phenyl, 4,6-dimethoxy-3-chloro-phenyl, pyridin-3-yl, pyridazin-3-yl, 6-chloro-pyridazin-3-yl or pyrazin-2-yl.

In a still further more preferred embodiment Ar¹ represents a phenyl group.

In a still further more preferred embodiment Ar¹ represents a pyridinyl group, in particular pyridin-3-yl, optionally substituted one or more times with alkyl, in particular methyl, and/or halo, in particular fluoro or chloro.

In a still further more preferred embodiment Ar¹ represents pyridin-3-yl, 6-fluoro-pyridin-3-yl, 2-fluoro-pyridin-3-yl, 6-chloro-pyridin-3-yl, 6-methyl-pyridin-3-yl, 2,5-difluoro-pyridin-3-yl, 2,6-difluoro-pyridin-3-yl or 2,5,6-trifluoro-pyridin-3-yl.

In a still further more preferred embodiment Ar¹ represents a pyridinyl group, in particular pyridin-3-yl.

In a still further more preferred embodiment Ar¹ represents 6-fluoro-pyridin-3-yl, 2-fluoro-pyridin-3-yl, 6-chloro-pyridin-3-yl or 6-methyl-pyridin-3-yl.

In a still further more preferred embodiment Ar¹ represents 2,5-difluoro-pyridin-3-yl, 2,6-difluoro-pyridin-3-yl or 2,5,6-trifluoro-pyridin-3-yl.

In a still further more preferred embodiment Ar¹ represents a pyridazinyl group, in particular pyridazin-3-yl, optionally substituted with halo, in particular chloro.

In a still further more preferred embodiment Ar¹ represents 6-chloro-pyridazin-3-yl.

In a still further more preferred embodiment Ar¹ represents a pyrimidinyl group, in particular pyrimidin-5-yl.

In a still further more preferred embodiment Ar¹ represents a pyrazinyl group, in particular pyridazin-3-yl.

In another preferred embodiment the compound of the invention is represented by Formula I, wherein Ar² represents alkyl-carbonyl-amino (acetamido), or a phenyl, furanyl, thienyl, isoxazolyl, thiazolyl or pyridinyl group, which phenyl, furanyl, thienyl, isoxazolyl, thiazolyl and pyridinyl groups may optionally be substituted one or more times with substituents selected from the group consisting of alkyl, halo, haloalkyl, haloalkoxy, nitro and cyano.

In a more preferred embodiment Ar² represents alkyl-carbonyl-amino (acetamido), or a phenyl, furanyl, thienyl, isoxazolyl, thiazolyl or pyridinyl group, which phenyl, furanyl, thienyl, isoxazolyl, thiazolyl and pyridinyl groups may optionally be substituted one or more times with substituents selected from the group consisting of alkyl, in particular methyl, halo, in particular fluoro, chloro or bromo, haloalkyl, in particular trifluoromethyl, nitro and cyano.

In another more preferred embodiment Ar² represents a phenyl, furanyl, thienyl, isoxazolyl, thiazolyl or pyridinyl group, which phenyl, furanyl, thienyl, isoxazolyl, thiazolyl and pyridinyl groups may optionally be substituted one or more times with substituents selected from the group consisting of alkyl, in particular methyl, halo, in particular fluoro, chloro or bromo, haloalkyl, in particular trifluoromethyl, nitro and cyano.

In an even more preferred embodiment Ar² represents alkyl-carbonyl-amino (acetamido), or a phenyl, furanyl, thienyl, isoxazolyl or pyridinyl group, which phenyl, furanyl, thienyl, isoxazolyl and pyridinyl groups may optionally be substituted one or more times with substituents selected from the group consisting of alkyl, halo, trifluoromethyl, trifluoromethoxy, nitro and cyano.

In a still more preferred embodiment Ar² represents alkyl-carbonyl-amino.

In a yet more preferred embodiment Ar² represents acetamido.

In another more preferred embodiment Ar² represents a phenyl, furanyl, thienyl, isoxazolyl or pyridinyl group, which phenyl, furanyl, thienyl, isoxazolyl and pyridinyl groups may optionally be substituted one or more times with substituents selected from the group consisting of alkyl, halo, trifluoromethyl, trifluoromethoxy, nitro and cyano.

In an even more preferred embodiment Ar² represents phenyl, optionally substituted one or two times with substituents selected from the group consisting of alkyl, halo, trifluoromethyl, nitro and cyano.

In a further more preferred embodiment Ar² represents acetamido, phenyl, 3-cyano-phenyl, 5-chloro-2-hydroxy-phenyl, 5-trifluoromethyl-2-chloro-phenyl, isoxazolyl, 5-methyl-isoxazol-3-yl or pyridinyl.

In a still further more preferred embodiment Ar² represents phenyl, 3-cyano-phenyl, 5-chloro-2-hydroxy-phenyl, 5-trifluoromethyl-2-chloro-phenyl, isoxazolyl, 5-methyl-isoxazol-3-yl or pyridinyl.

In a still further more preferred embodiment Ar² represents 3-cyano-phenyl, 4-methyl-3-bromo-phenyl, 5-chloro-2-hydroxy-phenyl, 5-trifluoromethyl-2-chloro-phenyl, 5-methyl-isoxazol-3-yl or pyridinyl.

In a still further more preferred embodiment Ar² represents furanyl or thienyl, optionally substituted with nitro, cyano or halo, in particular fluoro or bromo.

In a still further more preferred embodiment Ar² represents furanyl or thienyl, optionally substituted with nitro or cyano.

In a still further more preferred embodiment Ar² represents furanyl, 5-nitro-furan-2-yl; 5-nitro-furan-2-yl, 5-fluoro-furan-2-yl, 5-chloro-furan-2-yl or 5-bromo-furan-2-yl.

In a still further more preferred embodiment Ar² represents or thienyl, 5-cyano-thien-2-yl, 5-nitro-thien-2-yl, 5-bromo-thien-2-yl.

In a still further more preferred embodiment Ar² represents furanyl, in particular furan-2-yl or furan-3-yl, or thienyl, in particular thien-2-yl.

In a still further more preferred embodiment Ar² represents isoxazolyl, in particular isoxazol-3-yl or isoxazol-5-yl, optionally substituted with alkyl, in particular methyl.

In a still further more preferred embodiment Ar² represents isoxazol-3-yl, isoxazol-5-yl or 5-methyl-isoxazol-3-yl.

In a still further more preferred embodiment Ar² represents pyridinyl, in particular pyridin-3-yl, optionally substituted one or more times with halo, in particular fluoro or chloro.

In a still further more preferred embodiment Ar² represents pyridin-3-yl, 6-fluoro-pyridin-3-yl, 2-fluoro-pyridin-3-yl, 6-chloro-pyridin-3-yl, 6-methyl-pyridin-3-yl, 2,5-difluoro-pyridin-3-yl, 2,6-difluoro-pyridin-3-yl or 2,5,6-trifluoro-pyridin-3-yl.

In a still further more preferred embodiment Ar² represents thiazolyl, in particular thiazol-4-yl, optionally substituted with halo, in particular bromo.

In a still further more preferred embodiment Ar² represents pyridinyl, in particular pyridin-3-yl.

In a still further more preferred embodiment Ar² represents acetamido, phenyl, 3-nitro-phenyl, 3-cyano-phenyl, 5-chloro-2-hydroxy-phenyl, 5-trifluoromethyl-2-chloro-phenyl, 4-cyano-3-methyl-phenyl, 3-cyano-4-methyl-phenyl, 3-acetonitrile-phenyl, 3-carbamoyl-phenyl, furan-2-yl, 5-nitro-furan-2-yl, thien-2-yl, 5-cyano-thien-2-yl, isoxazol-3-yl, 5-methyl-isoxazol-3-yl or pyridine-3-yl.

In a still further more preferred embodiment Ar² represents phenyl, 3-nitro-phenyl, 3-cyano-phenyl, 5-chloro-2-hydroxy-phenyl, 5-trifluoromethyl-2-chloro-phenyl, 4-cyano-3-methyl-phenyl, 3-cyano-4-methyl-phenyl, 3-acetonitrile-phenyl, 3-carbamoyl-phenyl, furan-2-yl, 5-nitro-furan-2-yl, thien-2-yl, 5-cyano-thien-2-yl, isoxazol-3-yl, 5-methyl-isoxazol-3-yl or pyridine-3-yl.

In a still further more preferred embodiment Ar² represents pyrimidinyl, in particular pyrimidin-5-yl.

In a third preferred embodiment the compound of the invention is represented by Formula I, wherein L₁ and may be absent (i.e. represents a single covalent bond) or present, and if present represents a linking group selected from CH₂, CH₂CH₂, S, S—CH₂, O, O—CH₂, SO₂ and SO₂CH₂.

In a more preferred embodiment L₁ is absent (i.e. represents a single covalent bond).

In another more preferred embodiment L₁ is present, and represents a linking group selected from CH₂, CH₂CH₂, S, S—CH₂, O, O—CH₂; SO₂ and SO₂CH₂.

In an even more preferred embodiment L₁ is present, and represents a linking group selected from S, S—CH₂, and SO₂CH₂.

In a still more preferred embodiment L₁ represents S.

In a still further more preferred embodiment L₁ represents S—CH₂.

In a still further more preferred embodiment L₁ represents SO₂CH₂.

In a fourth preferred embodiment the compound of the invention is represented by Formula I, wherein X represents O or S.

In a more preferred embodiment X represents O.

In another more preferred embodiment X represents S.

In a fifth preferred embodiment the compound of the invention is represented by Formula I, wherein Ar¹ represents phenyl, optionally substituted one or more times with substituents selected from the group consisting of alkyl, hydroxy, alkoxy, halo, nitro, cyano, acetonitrile and amino-carbonyl (carbamoyl); and Ar² represents acetamido, phenyl, isoxazolyl or pyridinyl substituted once or twice with alkyl, halo, trifluoromethyl and/or cyano.

In a more preferred embodiment Ar¹ represents phenyl, optionally substituted one or more times with substituents selected from the group consisting of alkyl, hydroxy, alkoxy, halo, nitro, cyano, acetonitrile and amino-carbonyl (carbamoyl); and Ar² represents phenyl, isoxazolyl or pyridinyl substituted once or twice with alkyl, halo, trifluoromethyl and/or cyano.

In an even more preferred embodiment Ar¹ represents phenyl, 3-nitro-phenyl, 3-cyano-phenyl, 4-cyano-3-alkyl-phenyl, 3-cyano-4-alkyl-phenyl, 3-acetonitrile-phenyl, 5-chloro-2-hydroxy-phenyl, 5-trifluoromethyl-2-chloro-phenyl, 3-carbamoyl-phenyl or 4,6-dimethoxy-3-chloro-phenyl; and Ar² represents acetamido, phenyl, 3-cyano-phenyl, 5-chloro-2-hydroxy-phenyl, 5-trifluoromethyl-2-chloro-phenyl, isoxazolyl, 5-methyl-isoxazol-3-yl or pyridinyl.

In a still more preferred embodiment Ar¹ represents phenyl, 3-nitro-phenyl, 3-cyano-phenyl, 4-cyano-3-alkyl-phenyl, 3-cyano-4-alkyl-phenyl, 3-acetonitrile-phenyl, 5-chloro-2-hydroxy-phenyl, 5-trifluoromethyl-2-chloro-phenyl, 3-carbamoyl-phenyl or 4,6-dimethoxy-3-chloro-phenyl; and Ar² represents phenyl, 3-cyano-phenyl, 5-chloro-2-hydroxy-phenyl, 5-trifluoromethyl-2-chloro-phenyl, isoxazolyl, 5-methyl-isoxazol-3-yl or pyridinyl.

In a sixth preferred embodiment the compound of the invention is represented by Formula I, wherein Ar¹ represents a pyridinyl group; and Ar² represents phenyl, furanyl, thienyl or pyridinyl, which phenyl, furanyl, thienyl and pyridinyl are optionally substituted once or twice with alkyl, nitro and/or cyano.

In a more preferred embodiment Ar¹ represents a pyridinyl group; and Ar² represents phenyl, 3-nitro-phenyl, 3-cyano-phenyl, 4-cyano-3-methyl-phenyl, 3-cyano-4-methyl-phenyl, 3-acetonitrile-phenyl, 3-carbamoyl-phenyl, furanyl, 5-nitro-furan-2-yl, thienyl, 5-cyano-thien-2-yl or pyridinyl.

In an even more preferred embodiment Ar¹ represents a pyridinyl group; and Ar² represents phenyl or thienyl, which phenyl and thienyl are optionally substituted with alkyl, nitro and/or cyano.

In a still more preferred embodiment Ar¹ represents a pyridin-3-yl group; and Ar² represents phenyl or thienyl, which phenyl or thienyl is substituted with cyano.

In a seventh preferred embodiment the compound of the invention is represented by Formula I, wherein Ar¹ represents a pyridazinyl group, optionally substituted with halo; and Ar² represents a furanyl group.

In a more preferred embodiment Ar¹ represents 6-chloro-pyridazin-3-yl group; and Ar² represents a furan-2-yl group.

In an eight preferred embodiment the compound of the invention is represented by Formula I, wherein Ar¹ represents a pyrazinyl group; and Ar² represents a furanyl group.

In a more preferred embodiment Ar¹ represents a pyrazin-2-yl group; and

Ar² represents a furan-2-yl group.

In a most preferred embodiment the compound of the invention is
2-(5-Furan-2-yl-[1,3,4]oxadiazol-2-ylsulfanyl)-pyrazine;
3-(5-Benzylsulfanyl-[1,3,4]oxadiazol-2-yl)-pyridine;
3-Chloro-6-(5-furan-2-yl-[1,3,4]oxadiazol-2-ylsulfanyl)-pyridazine;
N-(5-Phenylmethanesulfonyl-[1,3,4]thiadiazol-2-yl)-acetamide;
3-[5-(5-Nitro-furan-2-yl)-[1,3,4]oxadiazol-2-yl]-pyridine;
3-[5-(3-Nitro-phenyl)-[1,3,4]oxadiazol-2-yl]-pyridine;
3-(5-Pyridin-3-yl-[1,3,4]oxadiazol-2-yl)-benzonitrile;
5-[5-(3-Cyano-phenyl)-[1,3,4]oxadiazol-2-yl]-benzonitrile;
5-[5-(3-Cyano-phenyl)-[1,3,4]thiadiazol-2-yl]-benzonitrile;
5-(5-Pyridin-3-yl-[1,3,4]oxadiazol-2-yl)-pyridine;
3-(5-Pyridin-3-yl-[1,3,4]thiadiazol-2-yl)-benzonitrile;
5-(5-Pyridin-3-yl-[1,3,4]oxadiazol-2-yl)-thiophene-2-carbonitrile;
2-Methyl-4-(5-pyridin-3-yl-[1,3,4]oxadiazol-2-yl)-benzonitrile;
2-Methyl-5-(5-pyridin-3-yl-[1,3,4]oxadiazol-2-yl)-benzonitrile;
[3-(5-Pyridin-3-yl-[1,3,4]oxadiazol-2-yl)-phenyl]-acetonitrile;
5-(5-Pyridin-3-yl-[1,3,4]thiadiazol-2-yl)-thiophene-2-carbonitrile;
4-Chloro-2-[5-(2-chloro-5-trifluoromethyl-phenyl)-[1,3,4]oxadiazol-2-yl]-phenol;
4-Chloro-2-[5-(2-chloro-5-trifluoromethyl-phenyl)-[1,3,4]thiadiazol-2-yl]-phenol;
3-(5-Pyridin-3-yl-[1,3,4]oxadiazol-2-yl)-benzamide;
2-(5-Chloro-2,4-dimethoxy-phenyl)-5-(5-methyl-isoxazol-3-yl)-[1,3,4]oxadiazole;
3-(5-Pyridin-3-yl-[1,3,4]oxadiazol-2-yl)-benzonitrile;
3-(5-{6-Chloro-pyridin-3-yl}-[1,3,4]oxadiazol-2-yl)-benzonitrile;
(3-[5-{6-Chloro-pyridin-3-yl}-[1,3,4]oxadiazol-2-yl]-phenyl)-acetonitrile;
3-(5-{6-Fluoro-pyridin-3-yl}-[1,3,4]oxadiazol-2-yl)-benzonitrile;
(3-{5-[6-Fluoro-pyridin-3-yl]-[1,3,4]oxadiazol-2-yl}-phenyl)-acetonitrile;
3-(5-Benzo[1,3]dioxol-5-yl-[1,3,4]oxadiazol-2-yl)-pyridine;
3-(5-[5-Nitro-thiophen-2-yl]-[1,3,4]oxadiazol-2-yl)-pyridine;
3-(5-{5-Nitro-thiophen-2-yl}-[1,3,4]oxadiazole-2-yl)-pyridine;
3-(5-{5-Bromo-furan-2-yl}-[1,3,4]-oxadiazol-2-yl)-pyridine;
3-(5-{Furan-2-yl}-[1,3,4]oxadiazole-2-yl)-pyridine;
3-(5-Isoxazol-5-yl-[1,3,4]oxadiazole-2-yl)-pyridine;
3-(5-{2-Bromo-thiazol-4-yl}-[1,3,4]oxadiazole-2-yl)-pyridine;
3-(5-Furan-3-yl-[1,3,4]oxadiazole-2-yl)-pyridine;
5-(5-{5-Bromo-thiophen-2-yl}-[1,3,4]oxadiazole-2-yl)-2-chloro-pyridine;
5-(5-{5-Bromo-thiophen-2-yl}-[1,3,4]oxadiazole-2-yl)-2-fluoro-pyridine;
4-(5-{2,6-Difluoro-pyridin-3-yl}-[1,3,4]oxadiazole-2-yl)-benzonitrile;
4-(5-{2,5,6-Trifluoro-pyridin-2-yl}-[1,3,4]oxadiazole-2-yl)-benzonitrile;
3-(5-{2,6-Difluoro-pyridin-3-yl}-[1,3,4]oxadiazole-2-yl)-benzonitrile;
3-(5-{1,5,6-Trifluoro-pyridin-3-yl}-[1,3,4]oxadiazole-2-yl)-benzonitrile;
3-(5-{4-Chloro-phenyl}-[1,3,4]oxadiazole-2-yl)-2,6-difluoro-pyridine;
3-(5-{4-Chloro-phenyl}-[1,3,4]oxadiazole-2-yl)-2,5,6-trifluoro-pyridine;
3-(5-{5-Bromo-thiophen-2-yl)-[1,3,4]oxadiazole-2-yl)-2-fluoro-pyridine;
3-(5-{2-Fluoro-pyridin-3-yl}-[1,3,4]oxadiazole-2-yl)-benzonitrile;
2,3,6-Trifluoro-5-(5-{3-fluoro-phenyl}-[1,3,4]oxadiazole-2-yl)-pyridine;
2,5-Difluoro-3-(5-{3-fluoro-phenyl}-[1,3,4]oxadiazole-2-yl)-pyridine;
3-(5-{4-Chlorophenyl}-[1,3,4]oxadiazole-2-yl)-2,5,difluoropyridine;
4-(5-{2,5-Difluoro-pyridine-3-yl}-[1,3,4]oxadiazole-2-yl)-benzonitrile;
2-(3-Bromo-4-methyl-phenyl)-5-(4-chloro-phenyl)-[1,3,4]oxadiazole;
4-(5-{3-Bromo-4-methyl-phenyl}-[1,3,4]oxadiazole-2-yl)-benzonitrile;
2-(3-Bromo-4-methyl-phenyl)-5-(3-fluoro-phenyl)-[1,3,4]oxadiazole;
3-(5-{3-Bromo-4-methyl-phenyl}-[1,3,4]oxadiazole-2-yl)-benzonitrile;
4-(5-{2-Fluoro-pyridin-3-yl}-[1,3,4]oxadiazole-2-yl)-benzonitrile;
2-Fluoro-3-(-{3-fluoro-phenyl}-[1,3,4]-oxadiazol-2-yl)-pyridine;
3-(5-{4-Chloro-phenyl}-[1,3,4]oxadiazole-2-yl)-2-fluoro-pyridine;
4-(5-{6-Fluoro-pyridin-3-yl}-[1,3,4]-2-yl)-benzonitrile;
5-(5-{4-Chloro-phenyl}-[1,3,4]oxadiazole-2-yl)-2-fluoro-pyridine; 2-Fluoro-5-(5-{3-fluoro-phenyl}-[1,3,4]oxadiazole-2-yl)-pyridine;
(3-{5-[2,5,6-Trifluoro-pyridine-3-yl]-[1,3,4]oxadiazole-2-yl}-phenyl)-acetonitrile;
2-(2-Fluoro-phenyl)-5-isoxazol-5-yl-[1,3,4]oxadiazole;
3-(5-{6-Methyl-pyridin-3-yl}-[1,3,4]oxadiazole-2-yl)-benzonitrile;
3-(5-Pyrimidin-5-yl-[1,3,4]oxadiazole-2-yl)-benzonitrile;
5-(5-{3-Fluorophenyl}-[1,3,4]oxadiazole-2-yl)-pyrimidine;
3-(5-{2,3-Dihydrobenzo-[1,4]dioxin-6-yl-[1,3,4]oxadiazole-2-yl)-pyridine;
3-(5-Isoxazol-5-yl-[1,3,4]oxadiazole-2-yl)-benzonitrile; or
2-Fluoro-3-(5-furan-3-yl-[1,3,4]oxadiazole-2-yl)-pyridine;

any of its isomers or any mixture of isomers, or a pharmaceutically-acceptable addition salt thereof.

Any combination of two or more of the embodiments described herein is considered within the scope of the present invention.

### Definition of Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O—” group, wherein alkyl is as defined above. Examples of preferred alkoxy groups of the invention include methoxy and ethoxy.

In the context of this invention halo represents fluoro, chloro, bromo or iodo, and haloalkyl group designates an alkyl group as defined herein, which alkyl group is substituted one or more times with halo. Thus a trihalomethyl group represents e.g. a trifluoromethyl group, a trichloromethyl group, and similar trihalo-substituted methyl groups. Preferred haloalkyl groups of the invention include trihalogenmethyl, preferably —CF₃.

In the context of this invention a haloalkoxy group designates an alkoxy group as defined herein, which alkoxy group is substituted one or more times with halo. Preferred haloalkoxy groups of the invention include trihalogenmethoxy, preferably —OCF₃.

### Pharmaceutically Acceptable Salts

The oxadiazole derivative of the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydro-chloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a compound of the invention include alkali metal salts, such as the sodium salt of a compound of the invention containing a carboxy group.

In the context of this invention the “onium salts” of N-containing compounds may also be contemplated as pharmaceutically acceptable salts. Preferred “onium salts” include the alkyl-onium salts, the cycloalkyl-onium salts, and the cycloalkylalkyl-onium salts. Particularly preferred onium salts of the invention include those created at the N-position according to the following Formula I′

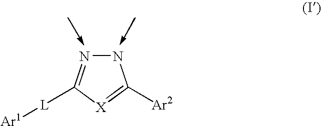

### Steric Isomers

It will be appreciated by those skilled in the art that the compounds of the present invention may exist in different stereoisomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L-(tartrates, mandelates or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials or intermediates.

### Methods of Producing Oxadiazole Derivatives

The oxadiazole derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision modulators of the nicotinic receptors, which modulators are useful for the treatment of diseases or disorders related to the nicotinic acetylcholine receptor (nAChR). Preferred compounds of the invention show a positive allosteric modulation of the nicotinic acetylcholine α4β2 receptor subtypes.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and abuse liability and withdrawal symptoms caused by the termination of abuse of chemical substances, in particular nicotine.

In a preferred embodiment the disease, disorder or condition relates to the central nervous system.

The compounds of the invention may also be useful as diagnostic tools or monitoring agents in various diagnostic methods, and in particular for in vivo receptor imaging (neuroimaging), and they may be used in labelled or unlabelled form.

In another preferred embodiment the disease, disorder or condition is a cognitive disorder, learning deficit, memory deficits and dysfunction, Down's syndrome, Alzheimer's disease, attention deficit, attention deficit hyperactivity disorder (ADHD), Tourette's syndrome, psychosis, depression, bipolar disorder, mania, manic depression, schizophrenia, cognitive or attention deficits related to schizophrenia, obsessive compulsive disorders (OCD), panic disorders, eating disorders such as anorexia nervosa, bulimia and obesity, narcolepsy, nociception, AIDS-dementia, senile dementia, autism, Parkinson's disease, Huntington's disease, amyotrophic lateral sclerosis (ALS), anxiety, non-OCD anxiety disorders, convulsive disorders, convulsions, epilepsy, neurodegenerative disorders, transient anoxia, induced neuro-degeneration, neuropathy, diabetic neuropathy, periferic dyslexia, tardive dyskinesia, hyperkinesia, pain, mild pain, moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury, bulimia, post-traumatic syndrome, social phobia, sleeping disorders, pseudodementia, Ganser's syndrome, pre-menstrual syndrome, late luteal phase syndrome, chronic fatigue syndrome, mutism, trichotillomania, jet-lag, arrhythmias, smooth muscle contractions, angina pectoris, premature labour, diarrhoea, asthma, tardive dyskinesia, hyperkinesia, premature ejaculation, erectile difficulty, hypertension, inflammatory disorders, inflammatory skin disorders, acne, rosacea, Chron's disease, inflammatory bowel disease, ulcerative colitis, diarrhoea, or abuse liability and withdrawal symptoms caused by termination of use of addictive substances, including nicotine containing products such as tobacco, opioids such as heroin, cocaine and morphine, benzodiazepines and benzodiazepine-like drugs, and alcohol.

In a more preferred embodiment the compounds of the invention are used for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In another more preferred embodiment the compounds of the invention are used for the treatment, prevention or alleviation of smooth muscle contractions, convulsive disorders, angina pectoris, premature labour, convulsions, diarrhoea, asthma, epilepsy, tardive dyskinesia, hyperkinesia, premature ejaculation, or erectile difficulty.

In a third more preferred embodiment the compounds of the invention are used for the treatment, prevention or alleviation of a neurodegenerative disorder, transient anoxia, or induced neuro-degeneration.

In a fourth more preferred embodiment the compounds of the invention are used for the treatment, prevention or alleviation of an inflammatory disorder, inflammatory skin disorder, acne, rosacea, Chron's disease, inflammatory bowel disease, ulcerative colitis, or diarrhoea.

In a fifth more preferred embodiment the compounds of the invention are used for the treatment, prevention or alleviation of diabetic neuropathy, schizophrenia, cognitive or attentional deficits related to schizophrenia, or depression.

In a sixth more preferred embodiment the compounds of the invention are used for the treatment, prevention or alleviation of pain, in particular neuropathic pain, diabetic neuropathy, schizophrenia and cognitive or attentional deficits related to schizophrenia, depression, and for assisting in obtaining smoking cessation.

In a seventh more preferred embodiment the compounds of the invention are used the treatment of abuse liability and withdrawal symptoms caused by termination of use of addictive substances, in particular nicotine containing products such as tobacco, opioids such as heroin, cocaine and morphine, cannabis, benzodiazepines, benzodiazepine-like drugs, and alcohol.

In an eight more preferred embodiment the compounds of the invention are used for the treatment of anxiety, cognitive disorders, learning deficit, memory deficits and dysfunction, Down's syndrome, Alzheimer's disease, attention deficit, attention deficit hyperactivity disorder (ADHD), Parkinson's disease, Huntington's disease, Amyotrophic Lateral Sclerosis, Gilles de la Tourette's syndrome, psychosis, depression, mania, manic depression, schizophrenia, obsessive compulsive disorders (OCD), panic disorders, eating disorders such as anorexia nervosa, bulimia and obesity, narcolepsy, nociception, AIDS-dementia, senile dementia, periferic neuropathy, autism, dyslexia, tardive dyskinesia, hyperkinesia, epilepsy, bulimia, post-traumatic syndrome, social phobia, sleeping disorders, pseudodementia, Ganser's syndrome, pre-menstrual syndrome, late luteal phase syndrome, chronic fatigue syndrome, mutism, trichotillomania, and jet-lag.

In a ninth more preferred embodiment the compounds of the invention are used for the treatment of cognitive disorders, psychosis, schizophrenia and/or depression.

In a tenth more preferred embodiment the compounds of the invention are used for the treatment of diseases, disorders, or conditions associated with smooth muscle contractions, including convulsive disorders, angina pectoris, premature labour, convulsions, diarrhoea, asthma, epilepsy, tardive dyskinesia, hyperkinesia, premature ejaculation, and erectile difficulty.

In an eleventh more preferred embodiment the compounds of the invention are used for the treatment of endocrine disorders, such as thyrotoxicosis, pheochromocytoma, hypertension and arrhythmias.

In a twelfth more preferred embodiment the compounds of the invention are used for the treatment of neurodegenerative disorders, including transient anoxia and induced neuro-degeneration.

In a thirteenth more preferred embodiment the compounds of the invention are used for the treatment of inflammatory diseases, disorders, or conditions, including inflammatory skin disorders such as acne and rosacea, Chron's disease, inflammatory bowel disease, ulcerative colitis, and diarrhoea.

In a fourteenth more preferred embodiment the compounds of the invention are used for the treatment of pain, mild, moderate or severe pain, or pain of acute, chronic or recurrent character, as well as pain caused by migraine, postoperative pain, and phantom limb pain. The pain may in particular be neuropathic pain, chronic headache, central pain, pain related to diabetic neuropathy, to post therapeutic neuralgia, or to peripheral nerve injury.

Finally, in a most preferred embodiment, the compounds of the invention may be useful for the treatment of depression, cognition, dementia, obesity, or associated with abuse liability and withdrawal symptoms caused by nicotine addiction.

In this context “treatment” covers treatment, prevention, prophylactics and alleviation of abuse liability and withdrawal symptoms and abstinence as well as treatment resulting in a voluntary diminished intake of the addictive substance.

In another aspect, the compounds of the invention are used as diagnostic agents, e.g. for the identification and localisation of nicotinic receptors in various tissues.

### Pharmaceutical Compositions

In another aspect the invention provides novel pharmaceutical compositions comprising a therapeutically effective amount of a oxadiazole or thiadiazole derivative of the invention.

In a preferred embodiment the pharmaceutical composition of the invention comprises a therapeutically effective amount of
N-(5-Benzylsulfanyl-[1,3,4]thiadiazol-2-yl)-acetamide;
3-(5-(5-Nitro-furan-2-yl)-[1,2,4]oxadiazol-3-yl)-pyridine;
3-(5-(3-Nitro-phenyl)-[1,2,4]oxadiazol-3-yl)-pyridine; or
2-Acetamido-5-benzylthio-[1,3,4]thiadiazole;

any of its isomers or any mixture of isomers, or a pharmaceutically-acceptable addition salt thereof.

While a compound of the invention for use in therapy may be administered in the form of the raw compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the oxadiazole derivative of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

In a preferred embodiment, when the pharmaceutical composition of the invention is intended for treating patients with abuse liability and withdrawal symptoms caused by nicotine addiction, formulations such as gums, patches, sprays, inhalers, aerosols, etc., are contemplated.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The oxadiazole derivatives of the present invention are valuable nicotinic and monoamine receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors and/or monoamine receptors, and which method comprises administering to such a living animal body, including a human, in need thereof an effective amount of an oxadiazole derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Preparatory Examples

### Example 1

### 5-Furan-2-yl-[1,3,4]oxadiazole-2-thiol (Intermediate compound)

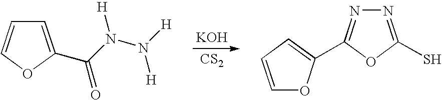

Potassium hydroxide 6.7 g (0.12 mole) was dissolved in 125 ml of methanol, 13.7 g (0.11 mole) of 2-furanoic hydrazide was added keeping the temperature at 25° C. for half an hour, then was 16.5 g (0.22 mole) of carbon disulfide added. The reaction mixture was heated to reflux and stirred for 8 hours, then evaporated to an oil. The residue was added water, concentrated hydrochloric acid was added until pH=4. The precipitate was isolated by filtration and dried. Yield 12.9 g (77 mmol, 70%).
In analogy herewith the following intermediate compound was made:
5-Pyridine-3-yl-[1,3,4]oxadiazole-2-thiol.

### Example 2

### 2-(5-Furan-2-yl-[1,3,4]oxadiazol-2-ylsulfanyl)-pyrazine (Compound 2.1)

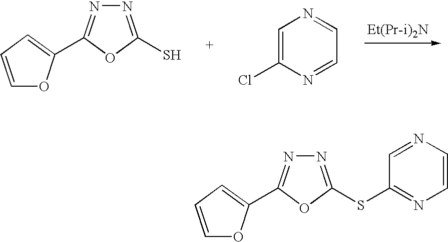

5-Furan-2-yl-[1,3,4]oxadiazole-2-thiol (1.8 g, 11 mmole) was dissolved in 50 ml of dry dioxane, to the solution was added 1.3 g (11 mmole) of chloropyrazine and 1.4 g (11 mmole) of ethyldiisopropyl amine. The reaction mixture was heated at reflux for 3 days and evaporated to an oil. The oil was added 1 N (aq.) sodium hydroxide, the product was isolated by filtration and purified by column chromatography. Yield 1.1 g (4.5 mmole, 41%); Mp. 92-93° C.
In analogy herewith the following compounds were made:
3-(5-Benzylsufanyl-[1,3,4]oxadiazol-2-yl)-pyridine (Compound 2.2)

Mp. 209-210° C.; and
3-Chloro-6-(5-furan-2-yl-[1.3.4]oxadiazol-2-ylsulfanyl)-pyridazine (Compound 2.3)

Mp. 132-133° C.

### Example 3

### N-(5-Phenylmethanesulfonyl-[1,3,4]oxadiazol-2-yl)-acetamide (Compound 3.1)

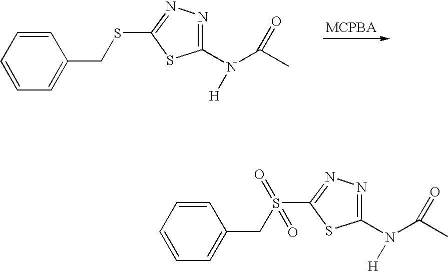

2-Acetamido-5-benzylthio-[1,3,4]thiadiazole (3 g, 11 mmole) in dichloromethane was added 8.6 g (50 mmole) of 3-chloroperbenzoic acid. The reaction mixture was stirred at room temperature for 90 minutes and filtrated. The filtrate was evaporated to an oil. The oil was triturated with water and a white precipitate was formed, which was isolated by filtration, the precipitate was washed with water and dried. Yield 2.6 g (8.7 mmole, 79%); Mp. 245-248° C.

### Example 4

### 5-Chloro-2,4-dimethoxy-benzoic acid ethyl ester (Intermediate compound)

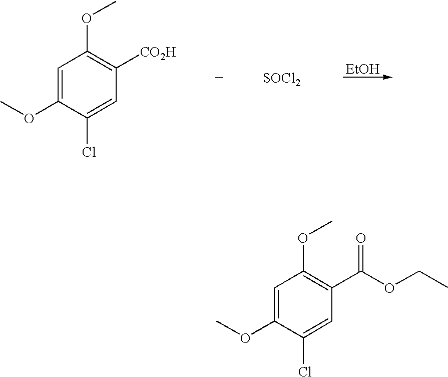

5-Chloro-2,4-dimethoxy-benzoic acid (1 g, 4.6 mmole) in 50 ml of ethanol (99%) was added 1.7 g (14 mmole) of thionyl chloride. The reaction mixture was stirred at 90° C. overnight and evaporated to an oil. The residue was added 100 ml of water and 300 ml of ethyl acetate. The organic phase was washed twice with 100 ml 10% sodium bicarbonate and twice with 100 ml of brine. The organic phase was dried and evaporated to dryness. Yield 1.1 g (4.5 mmole, 98%).

### Example 5

### 2-Chloro-2,4-dimethoxy-benzoic acid hydrazide (Intermediate compound)

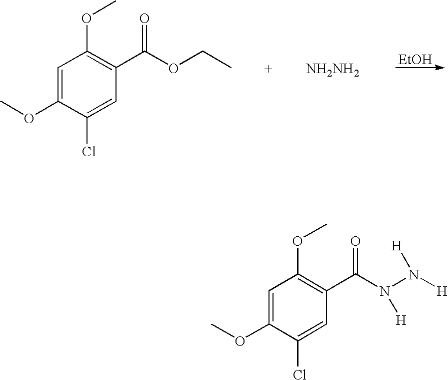

5-Chloro-2,4-dimethoxy-benzoic acid ethyl ester (1.1 g, 4.4 mmole) in 10 ml of ethanol (99%) was drop wise added to 3.2 ml (66 mmole) of hydrazine hydrate. The reaction mixture was stirred at room temperature for 15 minutes, then at 90° C. overnight. The reaction mixture was cooled to room temperature, added 100 ml of water and 300 ml of ethyl acetate. The water phase was extracted three times with 200 ml of ethyl acetate. The combined organic phases was washed, twice with 200 ml of 10% sodium bicarbonate, three times with 200 ml of brine, dried with sodium sulfate and evaporated to an oil, that was triturated with diethyl ether, the product was isolated by filtration. 0.72 g (3.1 mmole, 70%).

In analogy herewith the following intermediate compounds were made:
Nicotinic acid hydrazide;
3-Cyanobenzoic acid hydrazide;
3-Chloro-2-hydroxy-benzoic acid hydrazide;
3-Cyanobenzoic acid hydrazide;
3-Cyanomethylbenzoic acid hydrazide;
4-Cyanobenzoic acid hydrazide;
4-Chlorobenzoic acid hydrazide;
3-Fluorobenzoic acid hydrazide;
6-Methylnicotinic acid hydrazide.

### Example 6

### 3-Cyano-benzoic acid-N′-(pyridine-3-carbonyl)-hydrazide (Intermediate compound)

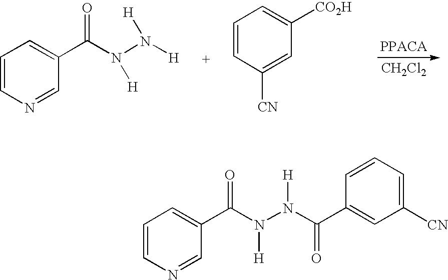

3-Cyano-benzoic acid (3.8 g, 25.5 mmole) and 10.7 ml (76.7 mmole) of triethyl amine in dichloromethane. The solution was cooled to 0° C. and 22.8 ml (76.7 mmole) of 1-propanephosphonic acid cyclic anhydride was added, stirring was continued for 20 minutes, nicotinic acid hydrazide was added, the reaction mixture was stirred at room temperature overnight and added brine. The organic layer was washed with saturated sodium bicarbonate solution, dried with sodium sulfate and evaporated to an oil. The product was purified by column chromatography. The product was used as this in the next step. Yield 2.6 g (9.8 mmole, 38%).

In analogy herewith the following intermediate compounds were made:
Nicotinic acid N′-(5-nitro-furan-2-carbonyl)-hydrazide;
Nicotinic acid N′-(3-nitro-benzoyl)-hydrazide;
Nicotinic acid N′-(3-cyano-benzoyl)-hydrazide;
3-Cyano-benzoic acid N′-(3-cyano-benzoyl)-hydrazide;
Nicotinic acid N′-(pyridine-3-carbonyl)-hydrazide;
5-Cyano-thiophene-2-carboxylic acid N′-(pyridine-3-carbonyl)-hydrazide;
4-Cyano-3-methyl-benzoic acid N′-(pyridine-3-carbonyl)-hydrazide;
3-Cyano-4-methyl-benzoic acid N′-(pyridine-3-carbonyl)-hydrazide;
3-Cyanomethyl-benzoic acid N′-(pyridine-3-carbonyl)-hydrazide;
3-[N′-(Pyridine-3-carbonyl)-hydrazinocarbonyl]-benzamide;
5-Methyl-isoxazole-3-carboxylic acid N′-(5-chloro-2,4-dimethoxy-benzoyl)-hydrazide;
4-Cyanobenzoic acid N′-(pyridine-3-carbonyl)-hydrazide;
3-Cyanobenzoic acid N′-(6-chloro-pyridine-3-carbonyl)-hydrazide;
4-Cyanomethylbenzoic acid N′(6-chloropyridine-3-carbonyl)-hydrazide;
3-Cyanobenzoic acid N′-(6-fluoropyridine-3-carbonyl)-hydrazide;
3-Cyanomethylbenzoic acid N′-(6-fluoro-pyridine-3-carbonyl)-hydrazide;
Benzo[1,3]dioxole-5-carboxylic acid N′-(pyridine-3-carbonyl)-hydrazide;
5-Nitro-thiophene-2-carboxylic acid N′-(pyridine-3-carbonyl)-hydrazide;
5-Chlorofuran-2-carboxylic acid N′-(pyridine-3-carbonyl)-hydrazide;
5-Bromofuran-2-carboxylic acid N′-(pyridine-3-carbonyl)-hydrazide;
Furan-2-carboxylic acid N′-(pyridine-3-carbonyl)-hydrazide;
Isoxazole-5-carboxylic acid N′-(pyridine-3-carbonyl)-hydrazide;
2-Bromothiazole-4-carboxylic acid N′-(pyridine-3-carbonyl)-hydrazide;
Furan-3-carboxylic acid N′-(pyridine-3-carbonyl)-hydrazide;
5-Bromothiophene-2-carboxylic acid N′-(6-chloro-pyridine-3-carbonyl)-hydrazide;
5-Bromothiophene-2-carboxylic acid N′-(6-fluoro-pyridine-3-carbonyl)-hydrazide;
4-Cyanobenzoic acid N′-(2,6-difluoropyridine-3-carbonyl)-hydrazide;
4-Cyanobenzoic acid N′-(2,5,6-trifluoropyridine-3-carbonyl)-hydrazide;
3-Cyanobenzoic acid N′-(2,6-difluoropyridine-3-carbonyl)-hydrazide;
3-Cyanobenzoic acid N′-(2,5,6-trifluoropyridine-3-carbonyl)-hydrazide;
4-Chlorobenzoic acid N′-(2,6-difluoropyridine-3-carbonyl)-hydrazide;
4-Chlorobenzoic acid N′-(2,5,6-trifluoropyridine-3-carbonyl)-hydrazide;
5-Bromothiophene-carboxylic acid N′-(2-fluoropyridine-3-carbonyl)-hydrazide;
3-Cyanobenzoic acid N′-(2-fluoropyridine-3-carbonyl)-hydrazide;
3-Bromo-4-methyl benzoic acid n′-(3-cyanobenzoyl)-hydrazide;
2-Fluoronicotinic acid N′-(4-cyanobenzoyl)-hydrazide;
2-Fluoronicotinic acid N′-(3-fluorobenzoyl)-hydrazide;
2-Fluoronicotinic acid N′-(4-chlorobenzoyl)-hydrazide;
6-Fluoronicotinic acid N′-(4-cyanobenzoyl)-hydrazide;
6-Fluoronicotinic acid N′-(4-chlorobenzoyl)-hydrazide;
6-Fluoronicotinic acid N′-(4-fluorobenzoyl)-hydrazide;
2,5,6-Trifluoronicotinic acid N′-(3-cyanobenzoyl)-hydrazide;
Isoxazole-5-carboxylic acid N′-(2-fluorobenzoyl)-hydrazide;
3-Cyanobenzoic acid N′-(6-methylpyridine-3-carbonyl)-hydrazide;
3-Cyanobenzoic acid N′-(pyrimidine-5-carbonyl)-hydrazide.

### Example 7

### 3-(5-Pyridine-3-yl-[1,3,4]oxadiazol-2-yl)-benzonitrile (Compound 7.1)

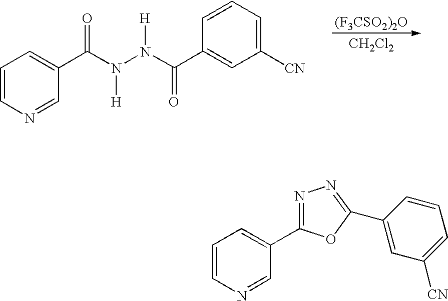

3-Cyano-benzoic acid-N′-(pyridine-3-carbonyl)-hydrazide (1.2 g, 4.5 mmole) in 25 ml of dichloromethane and 0.8 ml (9.5 mmole) of pyridine under a nitrogen atmosphere was cooled to −10° C., 2.8 g (9.9 mmole) of trifluoromethanesulfonic anhydride was added droop wise. The reaction mixture was stirred at −10° C. for one hour, then at 0° C. of one hour and at room temperature overnight. The reaction mixture was added 100 ml of 10% (aq.) sodium bicarbonate and 100 ml of dichloromethane. The organic phase was washed with 50 ml brine, dried with sodium sulfate and evaporated to an oil. The crude product was purified by column chromatography. Yield 0.6 g (2.4 mmole, 54%); Mp. 170-174° C.

In analogy herewith the following compounds were made:
3-(5-{5-Nitro-furan-2-yl}-[1,3,4]oxadiazol-2-yl)-pyridine (Compound 7.2)

Mp. 165-168° C.;
3-(5-{3-Nitro-phenyl}-[1,3,4]oxadiazol-2-yl)-pyridine (Compound 7.3)

Mp. 178-180° C.;
3-(5-{3-Cyano-phenyl}-[1,3,4]oxadiazol-2-yl)-benzonitrile (Compound 7.4)

Mp. 249-251° C.;
3-(5-Pyridin-3-yl-[1,3,4]oxadiazol-2-yl)-pyridine (Compound 7.5)

Mp. 181-186° C.;
3-(5-Pyridin-3-yl-[1,3,4]oxadiazol-2-yl)-thiophene-2-carbonitrile (Compound 7.6)

Mp. 218-234° C.;
2-Methyl-4-(5-pyridin-3-yl-[1,3,4]oxadiazol-2-yl)-benzonitrile (Compound 7.7)

Mp. 231-244° C.;
2-Methyl-5-(5-pyridin-3-yl-[1,3,4]oxadiazol-2-yl)-benzonitrile (Compound 7.8)

Mp. 152-167° C.;
(3-{5-Pyridin-3-yl-[1,3,4]oxadiazol-2-yl}-phenyl)-acetonitrile (Compound 7.9)

Mp. 231-238° C.;
3-(5-Pyridin-3-yl-[1,3,4]oxadiazol-2-yl)-thiophene-2-carbonitrile (Compound 7.10)

Mp. 237-241° C.;
4-Chloro-2-(5-{2-chloro-5-trifluoromethyl-phenyl-}[1,3,4]oxadiazol-2-yl)-phenol (Compound 7.11)

Mp. 113-119° C.;
3-(5-Pyridin-3-yl-[1,3,4]oxadiazol-2-yl)benzamide (Compound 7.12)

Mp. 233-239° C.;
2-(5-Chloro-2,4-dimethoxy-phenyl)-5-(5-methyl-isoxazol-3-yl)-[1,3,4]oxadiazole (Compound 7.13)

Mp. 201-205° C.;
3-(5-Pyridin-3-yl-[1,3,4]oxadiazol-2-yl)-benzonitrile (Compound 7.14)

Mp. 229-238° C.;
3-(5-{6-Chloro-pyridin-3-yl}-[1,3,4]oxadiazol-2-yl)-benzonitrile (Compound 7.15)

Mp. 207-209° C.;
(3-[5-{6-Chloro-pyridin-3-yl}-[1,3,4]oxadiazol-2-yl]-phenyl)-acetonitrile (Compound 7.16)

Mp. 175-180° C.;
3-(5-{6-Fluoro-pyridin-3-yl}-[1,3,4]oxadiazol-2-yl)-benzonitrile (Compound 7.17)

Mp. 198-207° C.;
(3-{5-[6-Fluoro-pyridin-3-yl]-[1,3,4]oxadiazol-2-yl}-phenyl)-acetonitrile (Compound 7.18)

Mp. 141-143° C.;
3-(5-Benzo[1,3]-dioxol-5-yl-[1,3,4]oxadiazol-2-yl)-pyridine (Compound 7.19)

LC-ESI-HRMS of [M+H]+ shows 268.0713 Da. Calc. 268.072217 Da, dev. −3.4 ppm;
3-(5-[5-Nitro-thiophen-2-yl]-[1,3,4]oxadiazol-2-yl)-pyridine (Compound 7.20)

LC-ESI-HRMS of [M+H]+ shows 275.0242 Da. Calc. 275.023887 Da, dev. 1.1 ppm;
3-(5-{5-Nitro-thiophen-2-yl-}[1,3,4]oxadiazole-2-yl)-pyridine (Compound 7.21)

LC-ESI-HRMS of [M+H]+ shows 248.0218 Da. Calc. 248.02268 Da, dev. −3.5 ppm;
3-(5-{5-Bromo-furan-2-yl}-[1,3,4]-oxadiazol-2-yl)-pyridine (Compound 7.22)

LC-ESI-HRMS of [M+H]+ shows 291.9715 Da. Calc. 291.972165 Da, dev. −2.3 ppm;
3-(5-{Furan-2-yl}-[1,3,4]oxadiazole-2-yl)-pyridine (Compound 7.23)

LC-ESI-HRMS of [M+H]+ shows 214.0618 Da. Calc. 214.061652 Da, dev. 0.7 ppm;
3-(5-Isoxazol-5-yl-[1,3,4]oxadiazole-2-yl)-pyridine (Compound 7.24)

LC-ESI-HRMS of [M+H]+ shows 215.0564 Da. Calc. 215.056901 Da, dev. −2.3 ppm;
3-(5-{2-Bromo-thiazol-4-yl}-[1,3,4]oxadiazole-2-yl)-pyridine (Compound 7.25)

LC-ESI-HRMS of [M+H]+ shows 308.9455 Da. Calc. 308.94457 Da, dev. 3 ppm;
3-(5-Furan-3-yl-[1,3,4]oxadiazole-2-yl)-pyridine (Compound 7.26)

LC-ESI-HRMS of [M+H]+ shows 214.0621 Da. Calc. 214.061652 Da, dev. 2.1 ppm;
5-(5-{5-Bromo-thiophen-2-yl}-[1,3,4]oxadiazole-2-yl)-2-chloro-pyridine (Compound 7.27)

Mp. 223-225° C.;
5-(5-{5-Bromo-thiophen-2-yl}-[1,3,4]oxadiazole-2-yl)-2-fluoro-pyridine (Compound 7.28)

Mp. 185-186° C.;
4-(5-{2,6-Difluoro-pyridin-3-yl}-[1,3,4]oxadiazole-2-yl)-benzonitrile (Compound 7.29)

LC-ESI-HRMS of [M+H]+ shows 285.0596 Da. Calc. 285.058792 Da, dev. 2.8 ppm;
4-(5-{2,5,6-Trifluoro-pyridin-2-yl}-[1,3,4]oxadiazole-2-yl)-benzonitrile (Compound 7.30)

LC-ESI-HRMS of [M+H]+ shows 303.0505 Da. Calc. 303.04937 Da, dev. 3.7 ppm;
3-(5-{2,6-Difluoro-pyridin-3-yl}-[1,3,4]oxadiazole-2-yl)-benzonitrile (Compound 7.31)

LC-ESI-HRMS of [M+H]+ shows 285.0577 Da. Calc. 285.058792 Da, dev. −3.8 ppm;
3-(5-{1,5,6-Trifluoro-pyridin-3-yl}-[1,3,4]oxadiazole-2-yl)-benzonitrile (Compound 7.32)

LC-ESI-HRMS of [M+H]+ shows 303.0506 Da. Calc. 303.04937 Da, dev. 4.1 ppm;
3-(5-{4-Chloro-phenyl}-[1,3,4]oxadiazole-2-yl)-2,6-difluoro-pyridine (Compound 7.33)

LC-ESI-HRMS of [M+H]+ shows 294.0258 Da. Calc. 294.024571 Da, dev. 4.2 ppm;
3-(5-{4-Chloro-phenyl}-[1,3,4]oxadiazole-2-yl)-2,5,6-trifluoro-pyridine (Compound 7.34)

LC-ESI-HRMS of [M+H]+ shows 312.0159 Da. Calc. 312.015149 Da, dev. 2.4 ppm;
3-(5-{5-Bromo-thiophen-2-yl)-[1,3,4]oxadiazole-2-yl)-2-fluoro-pyridine (Compound 7.35)

Mp. 188-190° C.;
3-(5-{2-Fluoro-pyridin-3-yl}-[1,3,4]oxadiazole-2-yl)-benzonitrile (Compound 7.36)

Mp. 171-173° C.;
2,3,6-Trifluoro-5-(5-{3-fluoro-phenyl}-[1,3,4]oxadiazole-2-yl)-Pyridine (Compound 7.37)

LC-ESI-HRMS of [M+H]+ shows 296.0455 Da. Calc. 296.044699 Da, dev. 2.7 ppm;
2,5-Difluoro-3-(5-{3-fluoro-phenyl}-[1,3,4]oxadiazole-2-yl)-pyridine (Compound 7.38)

LC-ESI-HRMS of [M+H]+ shows 278.0554 Da. Calc. 278.054121 Da, dev. 4.6 ppm;
3-(5-{4-Chlorophenyl}-[1,3,4]oxadiazole-2-yl)-2,5,difluoropyridine (Compound 7.39)

LC-ESI-HRMS of [M+H]+ shows 294.025 Da. Calc. 294.024571 Da, dev. 1.5 ppm;
4-(5-{2,5-Difluoro-pyridine-3-yl}-[1,3,4]oxadiazole-2-yl)-benzonitrile (Compound 7.40)

LC-ESI-HRMS of [M+H]+ shows 285.0586 Da. Calc. 285.058792 Da, dev. −0.7 ppm;
2-(3-Bromo-4-methyl-phenyl)-5-(4-chloro-phenyl)-[1,3,4]oxadiazole (Compound 7.41)

LC-ESI-HRMS of [M+H]+ shows 348.9739 Da. Calc. 348.974329 Da, dev. −1.2 ppm;
4-(5-{3-Bromo-4-methyl-phenyl}-[3,4]oxadiazole-2-yl)-benzonitrile (Compound 7.42)

LC-ESI-HRMS of [M+H]+ shows 340.01 Da. Calc. 340.00855 Da, dev. 4.3 ppm;
2-(3-Bromo-4-methyl-phenyl)-5-(3-fluoro-phenyl)-[1,3,4]oxadiazole (Compound 7.43)

LC-ESI-HRMS of [M+H]+ shows 333.0021 Da. Calc. 333.003879 Da, dev. −5.3 ppm;
3-(5-{3-Bromo-4-methyl-phenyl}-[1,3,4]oxadiazole-2-yl)-benzonitrile (Compound 7.44)

LC-ESI-HRMS of [M+H]+ shows 340.0068 Da. Calc. 340.00855 Da, dev. −5.1 ppm;
4-(5-{2-Fluoro-pyridin-3-yl}-[1,3,4]oxadiazole-2-yl)-benzonitrile (Compound 7.45)

LC-ESI-HRMS of [M+H]+ shows 267.0668 Da. Calc. 267.068214 Da, dev. −5.3 ppm;
2-Fluoro-3-(-{3-fluoro-phenyl}-[1,3,4]-oxadiazol-2-yl)-pyridine (Compound 7.46)

LC-ESI-HRMS of [M+H]+ shows 260.0626 Da. Calc. 260.063543 Da, dev. −3.6 ppm;
3-(5-{4-Chloro-phenyl}-[1,3,4]oxadiazole-2-yl)-2-fluoro-pyridine (Compound 7.47)

LC-ESI-HRMS of [M+H]+ shows 276.0336 Da. Calc. 276.033993 Da, dev. −1.4 ppm;
4-(5-{6-Fluoro-pyridin-3-yl}-[1,3,4]-2-yl)-benzonitrile (Compound 7.48)

LC-ESI-HRMS of [M+H]+ shows 267.0691 Da. Calc. 267.068214 Da, dev. 3.3 ppm;
5-(5-{4-Chloro-phenyl}-[1,3,4]oxadiazole-2-yl)-2-fluoro-pyridine (Compound 7.49)

LC-ESI-HRMS of [M+H]+ shows 276.0352 Da. Calc. 276.033993 Da, dev. 4.4 ppm;
2-Fluoro-5-(5-{3-fluoro-phenyl}-[1,3,4]oxadiazole-2-yl)-pyridine (Compound 7.50)

LC-ESI-HRMS of [M+H]+ shows 260.0642 Da. Calc. 260.063543 Da, dev. 2.5 ppm;
(3-{5-[2,5,6-Trifluoro-pyridine-3-yl]-[1,3,4]oxadiazole-2-yl}-phenyl)-acetonitrile (Compound 7.51)

LC-ESI-HRMS of [M+H]+ shows 317.0643 Da. Calc. 317.06502 Da, dev. −2.3 ppm;
2-(2-Fluoro-phenyl)-5-isoxazol-5-yl-[1,3,4]oxadiazole (Compound 7.52)

LC-ESI-HRMS of [M+H]+ shows 232.0528 Da. Calc. 232.05223 Da, dev. 2.5 ppm;
3-(5-{6-Methyl-pyridin-3-yl}-[1,3,4]oxadiazole-2-yl)-benzonitrile (Compound 7.53)

Mp. 228-234° C.;
3-(5-Pyrimidin-5-yl-[1,3,4]oxadiazole-2-yl)-benzonitrile (Compound 7.54)

Mp. 265-272° C.;
5-(5-{3-Fluorophenyl}-[1,3,4]oxadiazole-2-yl)-pyrimidine (Compound 7.55)

LC-ESI-HRMS of [M+H]+ shows 243.0677 Da. Calc. 243.068214 Da, dev. −2.1 ppm;
3-(5-{2,3-Dihydrobenzo-[1,4]dioxin-6-yl-[1,3,4]oxadiazole-2-yl)-pyridine (Compound 7.56)

LC-ESI-HRMS of [M+H]+ shows 282.0873 Da. Calc. 282.087867 Da, dev. −2 ppm;
3-(5-Isoxazol-5-yl-[1,3,4]oxadiazole-2-yl)-benzonitrile (Compound 7.57)

LC-ESI-HRMS of [M+H]+ shows 239.0556 Da. Calc. 239.056901 Da, dev. −5.4 ppm; and
2-Fluoro-3-(5-furan-3-yl-[1,3,4]oxadiazole-2-yl)-Pyridine (Compound 7.58);

LC-ESI-HRMS of [M+H]+ shows 232.0518 Da. Calc. 232.05223 Da, dev. −1.9 ppm.

### Example 8

### 3-(5-Pyridin-3-yl-[1,3,4]thiadiazol-2-yl)-benzonitrile (Compound 8.1)

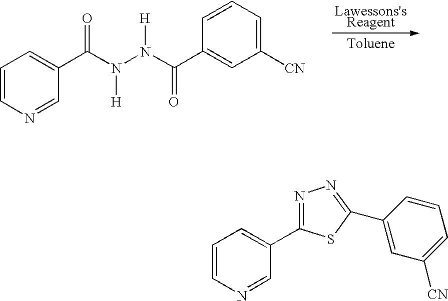

3-Cyano-benzoic acid-N′-(pyridine-3-carbonyl)-hydrazide (1.3 g, 4.7 mmole) in 40 ml of toluene was added 2.3 g (5.6 mmole), in a sealed container was the reaction mixture stirred at 100° C. overnight. The reaction mixture was evaporated, the residue was dissolved in 120 ml of ethyl acetate, this was washed with 50 ml of water, 30 ml of saturated brine, the organic phase was dried with sodium sulfate and evaporated to dryness. The residue was purified by column chromatography. Yield 1.1 g (4.2 mmole, 89%); Mp. 258-261° C.

In analogy herewith the following compounds were made:
3-(5-{3-Cyano-phenyl}-[1,3,4]thiadiazol-2-yl)-benzonitrile (Compound 8.2)

Mp. 217-223° C.; and
4-Chloro-2-(5-{2-chloro-5-trifluoromethyl-phenyl}-[1,3,4]thiadiazol-2-yl)-phenol (Compound 8.3);

Mp. 216-220° C.

### Example 9

### Biological Activity

Characterization of hα4β2 Positive Allosteric Modulators Using FLIPR

This experiment shows the modulating activity of compounds representative of the invention (i.e. 3-(5-Pyridin-3-yl-[1,3,4-]oxadiazol-2-yl)-benzonitrile, Compound 7.1; and 5-(5-Pyridin-3-yl-[1,3,4]oxadiazol-2-yl)-thiophene-2-carbonitrile, Compound 7.6) to positively modulate the response induced by a sub-maximal concentration of nicotine (EC₂₀₋₃₀) in human HEK-293 cells stably expressing the human nicotinic acetylcholine receptor subtype α4β2. The ability is determined relative to a maximal nicotine response (100 μM). The activity is determined as a fluorescence-based assay using a Fluorometric Imaging Plate Reader (FLIPR) as described below in more detail.

Full concentration/response curves are generated and EC₅₀ values are calculated based on peak values. EC₅₀ values (Effective Concentration) represent the concentration of the test substance, at which the nicotine-induced EC₂₀₋₃₀ response is positively modulated such that the size of the response equals 50% of the maximal response. The maximal positively modulated response (efficacy) is determined relative to the reference (nicotine) response.

Preferred compounds of the invention show an activity determined as EC₅₀ values in the low micro-molar range, preferably below 10 μM, more preferred in the sub-micromolar range, i.e. below 1 μM, and demonstrating a significant efficacy.

The results of this experiment are presented in Table 1 below.

TABLE 1
FLIPR nAChR α4β2 positive allosteric modulator activity

| | | Efficacy |
|---|---|---|
| | EC₅₀ | Response relative |
| Compound | (μM) | to Nicotine (%) |
| Compound 7.1 | 0.52 | 133 |
| Compound 7.6 | 0.98 | 153 |

1-15. (canceled)

16. A compound represented by Formula I 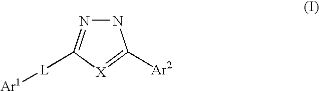
a stereoisomer or a mixture of its stereoisomers, an N-oxide, or a pharmaceutically acceptable addition salt thereof, wherein
Ar¹ represents a phenyl, pyridinyl, pyridazinyl, pyrimidinyl or pyrazinyl group, which phenyl, pyridinyl, pyridazinyl, pyrimidinyl and pyrazinyl groups may optionally be substituted one or more times with substituents selected from the group consisting of alkyl, hydroxy, alkoxy, halo, haloalkyl, haloalkoxy, nitro, cyano, acetonitrile, amino-carbonyl (carbamoyl) and methylenedioxy;
Ar² represents alkyl-carbonyl-amino (acetamido), or a phenyl, furanyl, thienyl, isoxazolyl, thiazolyl or pyridinyl group, which phenyl, furanyl, thienyl, isoxazolyl, thiazolyl and pyridinyl groups may optionally be substituted one or more times with substituents selected from the group consisting of alkyl, halo, haloalkyl, haloalkoxy, nitro and cyano;
L may be absent (i.e. represents a single covalent bond) or present, and if present represents a linking group selected from CH₂, CH₂CH₂, S, S—CH₂, O, O—CH₂, SO₂ and SO₂CH₂; and
X represents O or S.

17. The compound of claim 16, a stereoisomer or a mixture of its stereoisomers, an N-oxide, or a pharmaceutically acceptable addition salt thereof, wherein Ar¹ represents a phenyl, pyridinyl, pyridazinyl, pyrimidinyl or pyrazinyl group, which phenyl, pyridinyl, pyridazinyl, pyrimidinyl and pyrazinyl groups may optionally be substituted one or more times with substituents selected from the group consisting of alkyl, hydroxy, alkoxy, halo, haloalkyl, haloalkoxy, nitro, cyano, acetonitrile, amino-carbonyl (carbamoyl) and methylenedioxy.

18. The compound of claim 16, a stereoisomer or a mixture of its stereoisomers, an N-oxide, or a pharmaceutically acceptable addition salt thereof, wherein Ar² represents alkyl-carbonyl-amino (acetamido), or a phenyl, furanyl, thienyl, isoxazolyl, thiazolyl or pyridinyl group, which phenyl, furanyl, thienyl, isoxazolyl, thiazolyl and pyridinyl groups may optionally be substituted one or more times with substituents selected from the group consisting of alkyl, halo, haloalkyl, haloalkoxy, nitro and cyano.

19. The compound of claim 16, a stereoisomer or a mixture of its stereoisomers, an N-oxide, or a pharmaceutically acceptable addition salt thereof, wherein L₁ and may be absent (i.e. represents a single covalent bond) or present, and if present represents a linking group selected from CH₂, CH₂CH₂, S, S—CH₂, O, O—CH₂, SO₂ and SO₂CH₂.

20. The compound of claim 16, a stereoisomer or a mixture of its stereoisomers, an N-oxide, or a pharmaceutically acceptable addition salt thereof, wherein X represents O or S.

21. The compound of claim 16, a stereoisomer or a mixture of its stereoisomers, an N-oxide, or a pharmaceutically acceptable addition salt thereof, wherein
Ar¹ represents phenyl, optionally substituted one or more times with substituents selected from the group consisting of alkyl, hydroxy, alkoxy, halo, nitro, cyano, acetonitrile and amino-carbonyl (carbamoyl); and
Ar² represents acetamido, phenyl, isoxazolyl or pyridinyl, substituted once or twice with alkyl, halo, trifluoromethyl and/or cyano.

22. The compound of claim 16, a stereoisomer or a mixture of its stereoisomers, an N-oxide, or a pharmaceutically acceptable addition salt thereof, wherein
Ar¹ represents a pyridinyl group; and
Ar² represents phenyl, furanyl, thienyl or pyridinyl, which phenyl, furanyl, thienyl and pyridinyl groups are optionally substituted once or twice with alkyl, nitro and/or cyano.

23. The compound of claim 16, a stereoisomer or a mixture of its stereoisomers, an N-oxide, or a pharmaceutically acceptable addition salt thereof, wherein
Ar¹ represents a pyridazinyl group, optionally substituted with halo; and
Ar² represents a furanyl group.

24. The compound of claim 16, a stereoisomer or a mixture of its stereoisomers, an N-oxide, or a pharmaceutically acceptable addition salt thereof, wherein
Ar¹ represents a pyrazinyl group; and
Ar² represents a furanyl group.

25. The compound of claim 16, which is
2-(5-Furan-2-yl-[1,3,4]oxadiazol-2-ylsulfanyl)-pyrazine;
3-(5-Benzylsulfanyl-[1,3,4]oxadiazol-2-yl)-pyridine;
3-Chloro-6-(5-furan-2-yl-[1,3,4]oxadiazol-2-ylsulfanyl)-pyridazine;
N-(5-Phenylmethanesulfonyl-[1,3,4]thiadiazol-2-yl)-acetamide;
3-[5-(5-Nitro-furan-2-yl)-[1,3,4]oxadiazol-2-yl]-pyridine;
3-[5-(3-Nitro-phenyl)-[1,3,4]oxadiazol-2-yl]-pyridine;
3-(5-Pyridin-3-yl-[1,3,4]oxadiazol-2-yl)-benzonitrile;
5-[5-(3-Cyano-phenyl)-[1,3,4]oxadiazol-2-yl]-benzonitrile;
5-[5-(3-Cyano-phenyl)-[1,3,4]thiadiazol-2-yl]-benzonitrile;
5-(5-Pyridin-3-yl-[1,3,4]oxadiazol-2-yl)-pyridine;
3-(5-Pyridin-3-yl-[1,3,4]thiadiazol-2-yl)-benzonitrile;
5-(5-Pyridin-3-yl-[1,3,4]oxadiazol-2-yl)-thiophene-2-carbonitrile;
2-Methyl-4-(5-pyridin-3-yl-[1,3,4]oxadiazol-2-yl)-benzonitrile;
2-Methyl-5-(5-pyridin-3-yl-[1,3,4]oxadiazol-2-yl)-benzonitrile;
[3-(5-Pyridin-3-yl-[1,3,4]oxadiazol-2-yl)-phenyl]-acetonitrile;
5-(5-Pyridin-3-yl-[1,3,4]thiadiazol-2-yl)-thiophene-2-carbonitrile;
4-Chloro-2-[5-(2-chloro-5-trifluoromethyl-phenyl)-[1,3,4]oxadiazol-2-yl]-phenol;
4-Chloro-2-[5-(2-chloro-5-trifluoromethyl-phenyl)-[1,3,4]thiadiazol-2-yl]-phenol;
3-(5-Pyridin-3-yl-[1,3,4]oxadiazol-2-yl)-benzamide;
2-(5-Chloro-2,4-dimethoxy-phenyl)-5-(5-methyl-isoxazol-3-yl)-[1,3,4]oxadiazole;
3-(5-Pyridin-3-yl-[1,3,4]oxadiazol-2-yl)-benzonitrile;
3-(5-{6-Chloro-pyridin-3-yl}-[1,3,4]oxadiazol-2-yl)-benzonitrile;
(3-[5-{6-Chloro-pyridin-3-yl}-[1,3,4]oxadiazol-2-yl]-phenyl)-acetonitrile;
3-(5-{6-Fluoro-pyridin-3-yl}-[1,3,4]oxadiazol-2-yl)-benzonitrile;
(3-{5-[6-Fluoro-pyridin-3-yl]-[1,3,4]oxadiazol-2-yl}-phenyl)-acetonitrile;
3-(5-Benzo[1,3]dioxol-5-yl-[1,3,4]oxadiazol-2-yl)-pyridine;
3-(5-[5-Nitro-thiophen-2-yl]-[1,3,4]oxadiazol-2-yl)-pyridine;
3-(5-{5-Nitro-thiophen-2-yl}-[1,3,4]oxadiazole-2-yl)-pyridine;
3-(5-{5-Bromo-furan-2-yl}-[1,3,4]-oxadiazol-2-yl)-pyridine;
3-(5-{Furan-2-yl}-[1,3,4]oxadiazole-2-yl)-pyridine;
3-(5-Isoxazol-5-yl-[1,3,4]oxadiazole-2-yl)-pyridine;
3-(5-{2-Bromo-thiazol-4-yl}-[1,3,4]oxadiazole-2-yl)-pyridine;
3-(5-Furan-3-yl-[1,3,4]oxadiazole-2-yl)-pyridine;
5-(5-{5-Bromo-thiophen-2-yl}-[1,3,4]oxadiazole-2-yl)-2-chloro-pyridine;
5-(5-{5-Bromo-thiophen-2-yl}-[1,3,4]oxadiazole-2-yl)-2-fluoro-pyridine;
4-(5-{2,6-Difluoro-pyridin-3-yl}-[1,3,4]oxadiazole-2-yl)-benzonitrile;
4-(5-{2,5,6-Trifluoro-pyridin-2-yl}-[1,3,4]oxadiazole-2-yl)-benzonitrile;
3-(5-{2,6-Difluoro-pyridin-3-yl}-[1,3,4]oxadiazole-2-yl)-benzonitrile;
3-(5-{1,5,6-Trifluoro-pyridin-3-yl}-[1,3,4]oxadiazole-2-yl)-benzonitrile;
3-(5-{4-Chloro-phenyl}-[1,3,4]oxadiazole-2-yl)-2,6-difluoro-pyridine;
3-(5-{4-Chloro-phenyl}-[1,3,4]oxadiazole-2-yl)-2,5,6-trifluoro-pyridine;
3-(5-{5-Bromo-thiophen-2-yl)-[1,3,4]oxadiazole-2-yl)-2-fluoro-pyridine;
3-(5-{2-Fluoro-pyridin-3-yl}-[1,3,4]oxadiazole-2-yl)-benzonitrile;
2,3,6-Trifluoro-5-(5-{3-fluoro-phenyl}-[1,3,4]oxadiazole-2-yl)-pyridine;
2,5-Difluoro-3-(5-{3-fluoro-phenyl}-[1,3,4]oxadiazole-2-yl)-pyridine;
3-(5-{4-Chlorophenyl}-[1,3,4]oxadiazole-2-yl)-2,5,difluoropyridine;
4-(5-{2,5-Difluoro-pyridine-3-yl}-[1,3,4]oxadiazole-2-yl)-benzonitrile;
2-(3-Bromo-4-methyl-phenyl)-5-(4-chloro-phenyl)-[1,3,4]oxadiazole;
4-(5-{3-Bromo-4-methyl-phenyl}-[1,3,4]oxadiazole-2-yl)-benzonitrile;
2-(3-Bromo-4-methyl-phenyl)-5-(3-fluoro-phenyl)-[1,3,4]oxadiazole;
3-(5-{3-Bromo-4-methyl-phenyl}-[1,3,4]oxadiazole-2-yl)-benzonitrile;
4-(5-{2-Fluoro-pyridin-3-yl}-[1,3,4]oxadiazole-2-yl)-benzonitrile;
2-Fluoro-3-(-{3-fluoro-phenyl}-[1,3,4]-oxadiazol-2-yl)-pyridine;
3-(5-{4-Chloro-phenyl}-[1,3,4]oxadiazole-2-yl)-2-fluoro-pyridine;
4-(5-{6-Fluoro-pyridin-3-yl}-[1,3,4]-2-yl)-benzonitrile;
5-(5-{4-Chloro-phenyl}-[1,3,4]oxadiazole-2-yl)-2-fluoro-pyridine;
2-Fluoro-5-(5-{3-fluoro-phenyl}-[1,3,4]oxadiazole-2-yl)-pyridine;
(3-{5-[2,5,6-Trifluoro-pyridine-3-yl]-[1,3,4]oxadiazole-2-yl}-phenyl)-acetonitrile;
2-(2-Fluoro-phenyl)-5-isoxazol-5-yl-[1,3,4]oxadiazole;
3-(5-{6-Methyl-pyridin-3-yl}-[1,3,4]oxadiazole-2-yl)-benzonitrile;
3-(5-Pyrimidin-5-yl-[1,3,4]oxadiazole-2-yl)-benzonitrile;
5-(5-{3-Fluorophenyl}-[1,3,4]oxadiazole-2-yl)-pyrimidine;
3-(5-{2,3-Dihydrobenzo-[1,4]dioxin-6-yl-[1,3,4]oxadiazole-2-yl)-pyridine;
3-(5-Isoxazol-5-yl-[1,3,4]oxadiazole-2-yl)-benzonitrile; or
2-Fluoro-3-(5-furan-3-yl-[1,3,4]oxadiazole-2-yl)-pyridine;
a stereoisomer or a mixture of its stereoisomers, an N-oxide, or a pharmaceutically acceptable addition salt thereof.

26. A pharmaceutical composition comprising a therapeutically effective amount of a compound of claim 16, a stereoisomer or a mixture of its stereoisomers, an N-oxide, or a pharmaceutically acceptable addition salt thereof, together with at least one pharmaceutically acceptable carrier or diluent.

27. A method of treatment, prevention or alleviation of a disease or a disorder or a condition of a living animal body, including a human, which disorder, disease or condition is responsive to modulation of cholinergic receptors, which method comprises the step of administering to such a living animal body in need thereof a therapeutically effective amount of a compound of claim 16, a stereoisomer or a mixture of its stereoisomers, an N-oxide, or a pharmaceutically acceptable addition salt thereof.

28. The method according to claim 27, wherein the disease, disorder or condition is a cognitive disorder, learning deficit, memory deficits and dysfunction, Down's syndrome, Alzheimer's disease, attention deficit, attention deficit hyperactivity disorder (ADHD), Tourette's syndrome, psychosis, depression, bipolar disorder, mania, manic depression, schizophrenia, cognitive or attention deficits related to schizophrenia, obsessive compulsive disorders (OCD), panic disorders, eating disorders such as anorexia nervosa, bulimia and obesity, narcolepsy, nociception, AIDS-dementia, senile dementia, autism, Parkinson's disease, Huntington's disease, amyotrophic lateral sclerosis (ALS), anxiety, non-OCD anxiety disorders, convulsive disorders, convulsions, epilepsy, neurodegenerative disorders, transient anoxia, induced neuro-degeneration, neuropathy, diabetic neuropathy, peripheral dyslexia, tardive dyskinesia, hyperkinesia, pain, mild pain, moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury, bulimia, post-traumatic syndrome, social phobia, sleeping disorders, pseudodementia, Ganser's syndrome, pre-menstrual syndrome, late luteal phase syndrome, chronic fatigue syndrome, mutism, trichotillomania, jet-lag, arrhythmias, smooth muscle contractions, angina pectoris, premature labour, diarrhoea, asthma, tardive dyskinesia, hyperkinesia, premature ejaculation, erectile difficulty, hypertension, inflammatory disorders, inflammatory skin disorders, acne, rosacea, Crohn's disease, inflammatory bowel disease, ulcerative colitis, diarrhoea, or abuse liability and withdrawal symptoms caused by termination of use of addictive substances, including nicotine containing products such as tobacco, opioids such as heroin, cocaine and morphine, cannabis, benzodiazepines and benzodiazepine-like drugs, and alcohol.
